(19) United States
(12) Patent Application Publication
(10) Pub. No.: US 2025/0009566 A1
(43) Pub. Date: Jan. 9, 2025

(54) CUSTOMIZED EXTERNAL CRANIOPLASTY AND METHOD OF PRODUCTION

(71) Applicant: The Johns Hopkins University, Baltimore, MD (US)

(72) Inventors: Cristina Diana GHINDA, Baltimore, MD (US); William Stanley ANDERSON, Baltimore, MD (US); Jeffrey H. SIEWERDSEN, Baltimore, MD (US); Ryan STEWART, Baltimore, MD (US)

(73) Assignee: The Johns Hopkins University, Baltimore, MD (US)

(21) Appl. No.: 18/710,052

(22) PCT Filed: Nov. 16, 2022
(86) PCT No.: PCT/US2022/050108

§ 371 Date: May 14, 2024

Related U.S. Application Data
(60) Provisional application No. 63/280,484, filed on Nov. 17, 2021.

(51) Int. Cl. A61F 13/12; A61F 13/00; A61F 13/01

(52) U.S. Cl. CPC A61F 13/12; A61F 13/00987; A61F 13/01017; A61F 2013/00272

(57) ABSTRACT

A customized external cranioplasty includes a rigid section and a rim at least one of attached to or integral with the rigid section and extending at least partially around a periphery of the rigid section. The rigid section has a size and shape to substantially match a cranial defect and includes an impact-resistant material to provide protection and support to a user due to the cranial defect. The rim facilitates secure and comfortable contact of the customized external cranioplasty to the user's head. A method of producing a customized external cranioplasty includes receiving three-dimensional imaging data for at least a portion of a subject's skull that has a cranial defect: segmenting the cranial defect region from a surrounding portion of the subject's skull to obtain a surface requiring support by the customized external cranioplasty; and producing a rigid section of the customized external cranioplasty using said surface requiring support obtained from the segmenting.

### CROSS-REFERENCE TO RELATED APPLICATIONS

The present patent application claims priority benefit to U.S. Provisional Patent Application No. 63/280,484, filed on Nov. 17, 2021, the entire content of which is incorporated herein by reference. All references cited anywhere in this specification, including the Background and Detailed Description sections, are incorporated by reference as if each had been individually incorporated.

### BACKGROUND

### 1. Technical Field

Currently claimed embodiments of the invention relate to external cranioplasty, and more particularly to customized external cranioplasty devices and methods of production.

### 2. Discussion of Related Art

Traumatic brain injury (TBI) and strokes represent the leading cause of death and disability worldwide. According to the Centers for Disease Control and Prevention, in 2014 there were approximately 288,000 hospitalizations in the United States. The beneficial role of decompressive craniectomy for mass lesions and intracranial hypertension is well recognized in the setting of traumatic brain injury as well as in other pathologies such as large middle cerebral artery infarction. Those surgeries necessitate additional operations for cranial reconstruction and the patients could benefit from a customized external cranioplasty while waiting for the definitive cranioplasty. Decompressive craniectomy is a lifesaving procedure involving removal of a large skull area in order to accommodate the severe cerebral edema occurring in the setting of different pathologies such as severe TBI or middle cerebral artery infarction. The exposure of the brain to atmospheric pressure alters both intracranial hemodynamics and cerebrospinal fluid dynamics with secondary complications, such as the syndrome of the trephined. The severity of the symptoms fluctuates, and the incidence of the syndrome has been reported between 1% and 24% although some studies report a much larger proportion of affected patients. The definitive treatment represents surgery for cranioplasty; however, many patients cannot undergo a surgical intervention as the decision relies on the patient's neurological recovery, healing of the primary surgical wound, and lack of concurrent infection preventing a cranioplasty. These patients face a challenging recovery and a recent international consensus agreed that the cranioplasty may improve neurological function, and that an earlier cranioplasty may enhance this effect. Previous studies support an early cranioplasty to normalize the ICP and CSF flow which might be involved in the neurological and cognitive improvement observed in patients following cranioplasty. There is currently no protocol to prevent the clinical and radiological deterioration attributable to the syndrome of trephined and there are only case reports of a plaster molded to the patient's head for management of severe life-threatening syndrome of trephined. Thus, there remains a need for improved cranioplasty devices and methods of production.

### SUMMARY

A customized external cranioplasty according to an embodiment of the current invention includes a rigid section and a rim at least one of attached to or integral with the rigid section and extending at least partially around a periphery of the rigid section. The rigid section has a size and shape to substantially match a cranial defect and includes an impact-resistant material to provide protection and support to a user due to the cranial defect. The rim facilitates secure and comfortable contact of the customized external cranioplasty to the user's head.

A method of producing a customized external cranioplasty according to an embodiment of the current invention includes receiving three-dimensional imaging data for at least a portion of a subject's skull that has a cranial defect: segmenting the cranial defect region from a surrounding portion of the subject's skull to obtain a surface requiring support by the customized external cranioplasty; and producing a rigid section of the customized external cranioplasty using said surface requiring support obtained from the segmenting.

### BRIEF DESCRIPTION OF THE DRAWINGS

Embodiments of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

FIG. 1 shows several views of a customized external cranioplasty according to an embodiment of the current invention as custom fitted to a patient's skull.

FIG. 2 shows three views of the customized external cranioplasty of FIG. 1.

FIG. 3 shows three views of the customized external cranioplasty of FIG. 1 that further includes sensors.

FIG. 4 shows an example of customized external cranioplasty for a specific patient.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

A customized external cranioplasty according to an embodiment of the current invention can provide a non-invasive option for restoring the intracranial physiology in those challenging cases. Other embodiments can incorporate in the inset model, monitoring options such as electroencephalography or telemetric ICP sensors for comprehensive monitoring of the patient's neurological condition.

A large skull defect creates an intracranial and atmospheric pressure imbalance with secondary metabolic alterations as well as hydrodynamic and cerebral blood flow changes. A customized external cranioplasty according to some embodiments of the current invention represents a treatment option for patients with a previous craniectomy and a large skull defect for whom a surgical intervention is not possible.

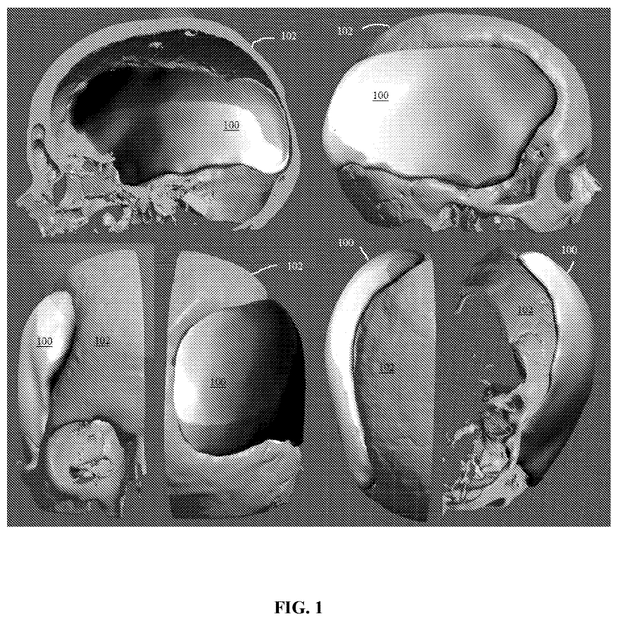

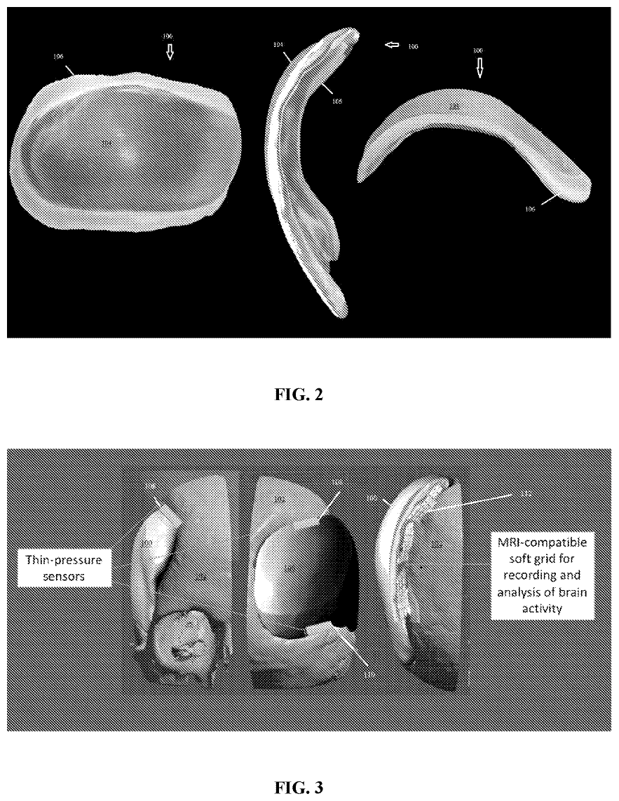

The customized external cranioplasty can be designed using the patient's CT scan that is segmented and converted to a 3D mesh. Although a CT scan is used in this example, other imaging modalities may be used according to other embodiments of the current invention. The mesh is subsequently used to design the cranioplasty model (FIG. 1). The inset model is designed to include a rim mesh using a softer flexible material (15%-Agilus30) that transitions with the defect margin in order to provide support around the edge of the skull defect. Although these materials were found to be suitable in this example, the broad concepts of the current invention are not limited to only these materials. Integrating a customized, patient-specific flexible material within the cranioplasty inset in order to facilitate the integration and biocompatibility of the bone-cranioplasty interface is a novel aspect of some embodiments of the current invention. The model, according to this embodiment, is subsequently printed using the 3D printer Stratasys Objet 260 using high resolution and rigid acrylic material (85% Vero) allowing a high impact resistance (FIG. 2). However, the broad concepts of the current invention are not limited to only the above-noted example of a suitable 3D printer. The customized insert can be incorporated in a protective headgear for the cranial defect with a customized protection that tries to restore the normal brain physiology according to some embodiments of the current invention. By creating a sealing effect customized to the patient's individual anatomy, this external cranioplasty aims to restore the equilibrium between intracranial and atmospheric pressure imbalance and thus is expected to prevent and/or improve the neurological deterioration of those patients. To our knowledge, the product represents the first 3D printed customized external cranioplasty option for management of skull deformities in patients who cannot undergo an immediate surgical intervention for the definitive cranioplasty.

FIG. 1 shows a customized external cranioplasty 100 that is customized to the skull 102 of a patient according to an embodiment of the current invention. The customized external cranioplasty 100 includes a rigid section 104 and a rim 106 at least one of attached to or integral with the rigid section 104 and extending at least partially around a periphery of the rigid section 104. (See FIG. 2.) The rigid section 104 has a size and shape to substantially match a cranial defect of the skull 102 and is made from a material that includes an impact-resistant material to provide protection and support to a user due to the cranial defect. The rim 106 facilitates secure and comfortable contact of the customized external cranioplasty 100 to the user's head.

The impact-resistant material can include, but is not limited to, a photopolymer. In some embodiments the photopolymer can include VeroClear and Agilus30. In some embodiments the photopolymer consists essentially of about 80% VeroClear and 20% Agilus30 by volume.

In some embodiments the rim includes Agilus30. In some embodiments the rim consists essentially of Agilus30.

In some embodiments the customized external cranioplasty further includes a sensor an example of which is shown in FIG. 3. The sensor can include, but is not limited to, at least one of a pressure sensor film 108, 110 or a conformal electrocorticography grid 112.

Another embodiment of the current invention is directed to a method of producing a customized external cranioplasty 100. The method of producing the customized external cranioplasty 100 includes receiving three-dimensional imaging data for at least a portion of a subject's skull 102 that has a cranial defect: segmenting the cranial defect region from a surrounding portion of the subject's skull 102 to obtain a surface requiring support by the customized external cranioplasty 100; and producing a rigid section 104 of the customized external cranioplasty using the surface requiring support obtained from the segmenting.

The three-dimensional imaging data can be, but is not limited to, at least one of x-ray CT imaging data or MRI data. The method of producing the rigid section 104 can include a three-dimensional printing of the rigid section 104 in some embodiments. In some embodiments, the method of producing the customized external cranioplasty 100 further includes producing a rim 106 extending around at least a portion of a periphery of the rigid section 104. The rim 106 can be at least one of attached to or integral with the rigid section 104. The producing of the rim can include a three-dimensional printing using the three-dimensional imaging data.

### Examples

### Background

Traumatic brain injury (TBI) and stroke represent the leading causes of death and disability worldwide. According to the Centers for Disease Control and Prevention, in 2014 there were approximately 288,000 hospitalizations in the United States^{1, 2}. Every year, 50 million people worldwide suffer from a TBI, and the annual global economic burden of TBI is estimated at approximately US $400 billion^{3, 4}.

The beneficial role of decompressive craniectomies for mass lesions and intracranial hypertension is well recognized for TBI as well as in other pathologies such as large middle cerebral artery infarctions. As a lifesaving procedure, a decompressive craniectomy involves the removal of a large skull area to accommodate the severe cerebral edema occurring as the result of different pathologies. Nonetheless, the exposure of the brain to atmospheric pressure alters both intracranial hemodynamics and cerebrospinal fluid dynamics with secondary complications, such as the syndrome of the trephined (SoT)⁵. The SoT is an important complication of craniectomy and is characterized by neurological dysfunction, including motor and cognitive deficits that improve with cranioplasty⁵. The reported SoT incidence varies between 1% and 24%^{6, 7}, though some studies report a much more significant proportion of affected patients⁸. The severity of the symptoms can fluctuate and have been associated with a prolonged time to cranioplasty, which represents the definitive treatment for SoT. However, many patients cannot undergo surgical intervention as the patient's neurological recovery, healing of the primary surgical wound, and presence of concurrent infection might prevent a cranioplasty.

Recently, rapid technological advancements have become the hallmark of the functional neurosurgery field, which has significantly expanded the therapeutic options for patients previously not deemed surgical candidates. For instance, novel closed-loop neuromodulation systems such as the RNS System (NeuroPace Inc, Mountain View, California) are currently utilized for patients with intractable epilepsy that have frequent and disabling partial seizures near eloquent areas of the brain with ≤2 foci of epileptogenesis. Nonetheless, the new techniques have associated complications, of which infection and skin erosion are most common⁹. The occurrence of an infection in the setting of previous instrumentation has significant implications requiring the explanation of a device that previously improved the patient's quality of life. Moreover, the bone removal and device explanation itself is not a minimal procedure and is associated with a significant risk of morbidity and mortality given the associated adhesions in the setting of multiple surgeries often performed in this patient population^{10, 11}.

These patients face a challenging recovery, and a recent international consensus agreed that the cranioplasty could improve neurological function, and an earlier procedure might enhance this effect¹². Previous studies support an early cranioplasty to normalize the ICP and CSF flow which might be involved in the neurological and cognitive improvement observed in patients following a cranioplasty¹³. There is currently no protocol to prevent the clinical and radiological deterioration attributed to the syndrome of the trephined, and there are only case reports of plaster molded to the patient's head for management of the severe life-threatening syndrome of the trephined^{14, 15}. This study reports the first experience with a novel non-invasive technique to manage and potentially prevent the SoT.

### Clinical Presentation

The patient was a 41-year-old woman with a past medical history of intractable epilepsy for which she underwent multiple right cerebral resections. The latest resection of a right temporal seizure focus was performed four years ago, and the patient was subsequently seizure-free (Engel class 1). Nonetheless, she unfortunately developed recurrent episodes of wound breakdown, CSF leak, and the loss of the cranioplasty reconstruction and titanium mesh reconstruction. In the setting of repeated wound dehiscence and exposed hardware, a right supine scalp wound revision with removal of the titanium mesh and a full-thickness skin graft from the abdomen was performed. Given her significant wound healing issues with repeated loss of her previous cranioplasty reconstructions due to recurrent bacterial and fungal infections, it was recommended by the plastics surgery consultant to avoid any foreign body implant for at least one year.

The patient had a good post-operative recovery with no signs of infection. However, she started to develop headaches localized on the left parietal-temporal area occurring predominantly in the supine position with no other focal neurological symptoms. The patient was also very concerned about her skull defect, and her symptoms were significantly affecting her quality of life. Given the lack of any focal neurological or infection signs, the patient's symptoms were considered part of the syndrome of the trephined. As there were no other surgical options, a customized external cranioplasty and helmet insert were designed after a CT scan was performed.

### Technique

The customized external cranioplasty was designed using the patient's CT scan after it was segmented and converted to a 3D mesh. The mesh was subsequently used to design the cranioplasty model (FIG. 1). The inset model is designed to include a rim mesh using a softer flexible material (15%-Agilus30) that transitions with the defect margin in order to provide support around the edge of the skull defect. To our knowledge, this is the first prototype integrating a customized, patient-specific flexible material within the cranioplasty inset to facilitate the bone-cranioplasty interface's integration and biocompatibility. The model was subsequently printed using the 3D Printer Stratasys Objet 260 using high resolution and rigid acrylic material (85% Vero), allowing a high impact resistance (FIG. 2). The customized inset can equally be incorporated in protective headgear for the cranial defect with customized protection that attempts to restore the normal brain physiology.

### Results

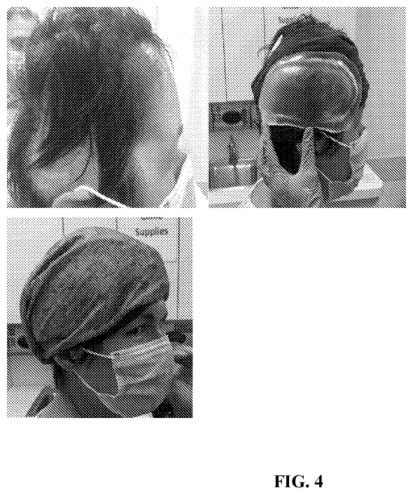

At the last follow-up (three months after the external cranioplasty fitting), the patient reported a significant improvement in her symptoms and quality of life. The patient reported a substantial reduction in her headaches and had no other focal neurological signs or symptoms. She achieved complete seizure control (Engel class 1) and had no recurrent infection symptoms. In addition, the method corrected her significant scalp/skull deformity, improving the cranial contour and optimizing patient satisfaction (FIG. 4).

### Discussion

A large skull defect creates an intracranial and atmospheric pressure imbalance with secondary metabolic alterations as well as hydrodynamic and cerebral blood flow changes^{5, 15}. Decompressive craniectomies necessitate additional operation for cranial reconstruction, and the patients could benefit from the customized external cranioplasty while waiting for the definitive cranioplasty. Our customized external cranioplasty represents a treatment option for patients with a previous craniectomy and a large skull defect for whom surgical intervention is not possible. Our customized external cranioplasty provides a safe, non-invasive option for restoring intracranial physiology in those challenging cases. This technique aims to restore the equilibrium between intracranial and atmospheric pressure imbalance and thus is expected to improve the patient's symptoms.

Currently, there are very limited options for treating patients suffering from the SoT when a surgical intervention cannot be performed. As such, this is a ground-breaking improvement to the management options of skull deformities in patients who cannot undergo surgical intervention for the definitive cranioplasty as there is no current method to prevent the trephined syndrome for patients where a cranial reconstruction cannot be performed immediately (i.e., due to recent injury, infection, etc.). Delayed cranioplasty after a decompressive craniectomy can result in SoT¹⁶ and seizures¹⁷, and our technique could equally represent a preventive measure to mitigate those devastating complications.

### Conclusion

To our knowledge, this study presents the first 3D printed customized external cranioplasty option for managing skull deformities in patients who cannot undergo surgical intervention for the definitive cranioplasty. By creating a sealing effect customized to the patient's individual anatomy, the CURE external cranioplasty can safely prevent and/or improve the neurological deterioration of those patients. In addition, other embodiments can incorporate the inset model monitoring options such as electroencephalography or telemetric intracranial pressure sensor for comprehensive monitoring of the patient's neurological condition.

### REFERENCES

1. Frieden T R, Houry D, Baldwin G: Centers for Disease Control and Prevention. (2014). Report to Congress on Traumatic Brain Injury in the United States: Epidemiology and Rehabilitation. National Center for Injury Prevention and Control; Division of Unintentional Injury Prevention. Atlanta 1-72, 2014
2. Defillo A: Letter to the Editor [Internet]. J Neurosurg 116:256-257, 2012 Available from: https://thejns.org/view/journals/j-neurosurg/116/1/article-p256.xml
3. Majdan M, Plancikova D, Brazinova A, et al: Epidemiology of traumatic brain injuries in Europe: a cross-sectional analysis [Internet]. Lancet Public Heal 1: e76-e83, 2016 Available from: http://dx.doi.org/10.1016/S2468-2667 (16) 30017-2
4. Maas A I R, Menon D K, Adelson P D, et al: Traumatic brain injury: integrated approaches to improve prevention, clinical care, and research [Internet]. Lancet Neurol 16:987-1048, 2017 Available from: https://linkinghub.elsevier.com/retrieve/pii/S147444221730371X
5. Ashayeri K, Jackson E M, Huang J, et al: Syndrome of the Trephined [Internet]. Neurosurgery 79:525-534, 2016 Available from: https://academic.oup.com/neurosurgery/article/79/4/525/2848935
6. Sedney C L, Dillen W, Julien T: Clinical spectrum and radiographic features of the syndrome of the trephined [Internet]. J Neurosci Rural Pract 6:438-441, 2015 Available from: http://www.thieme-connect.de/DOI/DOI?10.4103/0976-3147.158778
7. Honeybul S: Complications of decompressive craniectomy for head injury [Internet]. J Clin Neurosci 17:430-435, 2010 Available from: https://doi.org/10.1016/j.jocn.2009.09.007
8. Hagan M, Bradley J P: Syndrome of the Trephined [Internet]. J Craniofac Surg 28:1129-1130, 2017 Available from: http://journals.lww.com/00001665-201707000-00005
9. Wei Z, Gordon C R, Bergey G K, et al: Implant Site Infection and Bone Flap Osteomyelitis Associated with the NeuroPace Responsive Neurostimulation System [Internet]. World Neurosurg 88:687.e1-687.e6, 2016 Available from: https://linkinghub.elsevier.com/retrieve/pii/S1878875015017751
10. Jiménez-Martínez E, Cuervo G, Hornero A, et al: Risk factors for surgical site infection after craniotomy: a prospective cohort study [Internet]. Antimicrob Resist Infect Control 8:69, 2019 Available from: https://aricjournal.biomedcentral.com/articles/10.1186/s13756-019-0525-3
11. Krucoff M O, Chan A Y, Harward S C, et al: Rates and predictors of success and failure in repeat epilepsy surgery: A meta-analysis and systematic review [Internet]. Epilepsia 58:2133-2142, 2017 Available from: https://onlinelibrary.wiley.com/doi/10.1111/epi.13920
12. Iaccarino C, Kolias A, Adelson P D, et al: Consensus statement from the international consensus meeting on post-traumatic cranioplasty [Internet]. Acta Neurochir (Wien) 163:423-440, 2021 Available from: https://doi.org/10.1007/s00701-020-04663-5
13. Lilja-Cyron A, Andresen M, Kelsen J, et al: Intracranial pressure before and after cranioplasty: insights into intracranial physiology [Internet]. J Neurosurg 133:1548-1558, 2020 Available from: https://thejns.org/view/journals/j-neurosurg/133/5/article-p1548.xml
14. Dillen W L, Pittman T A, Grupke S L: Novel Temporary Treatment for a Severe Case of Syndrome of Trephined [Internet]. World Neurosurg 120:200-204, 2018 Available from: https://doi.org/10.1016/j.wneu.2018.08.153
15. Sarti T H M, de Araújo Paz D, Diniz J M, et al: External cranioplasty for the syndrome of the trephined-Case report [Internet]. Interdiscip Neurosurg 24:101065, 2021 Available from: https://linkinghub.elsevier.com/retrieve/pii/S2214751920306265
16. Gopalakrishnan M S, Shanbhag N C, Shukla D P, et al: Complications of Decompressive Craniectomy [Internet]. Front Neurol 9:5-7, 2018 Available from: https://www.frontiersin.org/article/10.3389/fneur.2018.00977/full
17. Shih F-Y, Lin C-C, Wang H-C, et al: Risk factors for seizures after cranioplasty [Internet]. Seizure 66:15-21, 2019 Available from: https://doi.org/10.1016/j.seizure.2018.12.016

While various embodiments of the present invention are described above, it should be understood that they are presented by way of example only, and not limitation. Thus, the breadth and scope of the present invention should not be limited by any of the described illustrative embodiments but should instead be defined only in accordance with the following claims and their equivalents.

The embodiments illustrated and discussed in this specification are intended only to teach those skilled in the art how to make and use the invention. In describing embodiments of the disclosure, specific terminology is employed for the sake of clarity. However, the disclosure is not intended to be limited to the specific terminology so selected. The above-described embodiments of the disclosure may be modified or varied, without departing from the invention, as appreciated by those skilled in the art in light of the teachings herein. It is therefore to be understood that, within the scope of the claims and their equivalents, the invention may be practiced otherwise than as specifically described. For example, it is to be understood that the present disclosure contemplates that, to the extent possible, one or more features of any embodiment can be combined with one or more features of any other embodiment.

1. A customized external cranioplasty, comprising:
a rigid section; and
a rim at least one of attached to or integral with said rigid section and extending at least partially around a periphery thereof,
wherein said rigid section has a size and shape to substantially match a cranial defect, said rigid section comprising an impact-resistant material to provide protection and support to a user due to said cranial defect,
wherein said rim facilitates secure and comfortable contact of said customized external cranioplasty to said user's head.

2. The customized external cranioplasty according to claim 1, wherein said impact-resistant material comprises a photopolymer.

3. The customized external cranioplasty according to claim 2, wherein said photopolymer comprises VeroClear and Agilus30.

4. The customized external cranioplasty according to claim 3, wherein said photopolymer consists essentially of about 80% VeroClear and 20% Agilus30 by volume.

5. The customized external cranioplasty according to claim 1, wherein a material of said rim comprises Agilus30.

6. The customized external cranioplasty according to claim 5, wherein said material of said rim consists essentially of Agilus30.

7. The customized external cranioplasty according to claim 1, further comprising a sensor.

8. The customized external cranioplasty according to claim 7, wherein said sensor is at least one of a pressure sensor film or a conformal electrocorticography grid.

9. A method of producing a customized external cranioplasty, comprising:
receiving three-dimensional imaging data for at least a portion of a subject's skull that has a cranial defect;
segmenting said cranial defect region from a surrounding portion of said subject's skull to obtain a surface requiring support by said customized external cranioplasty; and
producing a rigid section of said customized external cranioplasty using said surface requiring support obtained from said segmenting.

10. The method of claim 9, wherein said three-dimensional imaging data is at least one of x-ray CT imaging data or MRI data.

11. The method of claim 9, wherein said producing said rigid section comprises a three-dimensional printing of said rigid section.

12. The method of claim 9, further comprising producing a rim extending at least a portion around a periphery of said rigid section, said rim being at least one of attached to or integral with said rigid section.

13. The method of claim 12, wherein said producing said rim comprises a three-dimensional printing using said three-dimensional imaging data.

14. A customized external cranioplasty produced according to claim 9.
